(19) United States
(12) Patent Application Publication
(10) Pub. No.: US 2024/0285719 A1
(43) Pub. Date: Aug. 29, 2024

(54) FERMENTATION COMPLEX WITH DELAYING AGING AND IMPROVING SLEEPING EFFECT BY GENERATING OF BRAIN DOPAMINE, PREPARATION AND APPLICATION THEREOF

(71) Applicant: BIOZYME BIOTECHNOLOGY CORP., Banqiao City (TW)

(72) Inventors: CHENG HUANG, Banqiao City (TW); YI-CHUNG LAI, Banqiao City (TW); PO-JU LAI, Banqiao City (TW); PING-CHENG CHAN, Banqiao City (TW)

(21) Appl. No.: 18/587,236

(22) Filed: Feb. 26, 2024

(30) Foreign Application Priority Data
Feb. 24, 2023 (TW) 112107044

(51) Int. Cl. A61K 36/899; A61K 36/898; A61P 25/00; A61P 25/16; A61Q 19/08; C12P 1/04

(52) U.S. Cl. CPC A61K 36/899; A61K 36/898; A61P 25/00; A61P 25/16; A61Q 19/08; C12P 1/04; A61K 2236/15; A61K 2236/19; A61K 2236/30; A61K 2236/331

(57) ABSTRACT

A fermentation complex with increasing generation of brain dopamine and improving sleeping effect, the fermentation complex containing a vegetable ingredient and use a special polysaccharide fermentation preparation method to get a fermentation complex, within the vegetable ingredient include Gastrodia elata, Black rice, and Wheat seedlings. The fermentation complex has effect of delaying brain aging, protecting brain nerves, calming nerves, and helping to fall asleep.

### CROSS-REFERENCE TO RELATED APPLICATIONS

This application claims priority in Taiwan Patent Application No. 112107044, filed on Feb. 24, 2023, which is incorporated by reference in its entirety herein.

### FIELD OF THE INVENTION

The present invention relates to a novel fermented composite of Gastrodia elata, Black rice, and Wheat seedlings with the ability to delay aging and promote sleep through dopamine generation. This complex effectively stimulates the endogenous production of dopamine, glutamic acid, and vitamin B6 in the human body, thereby retarding brain aging, safeguarding neurological functions, and facilitating a calming effect on the nervous system to aid in the induction of sleep.

### BACKGROUND OF THE INVENTION

Dopamine stands as a pivotal neurotransmitter with profound implications for an individual's emotional state. This organic compound assumes multifaceted roles within both the brain and body, primarily synthesized within the human brain and kidneys. Within the brain, dopamine operates as a neurotransmitter, facilitating signal transmission between neurons through chemical release. Numerous significant neurological disorders are linked to dysfunctions within the dopamine system, necessitating the utilization of key medications capable of modulating dopamine activity in their therapeutic interventions.

For instance, Parkinson's disease, a degenerative condition characterized by tremors and movement impairments, is intricately linked to insufficient dopamine secretion by neurons within the substantia nigra region of the midbrain. Its metabolic precursor, L-DOPA, can be synthetically produced, with levodopa emerging as the predominant therapeutic modality. Evidence underscores schizophrenia's involvement with alterations in dopamine activity levels, with the majority of antipsychotic medications exerting their effects primarily through dopamine activity reduction. Additionally, Restless Legs Syndrome and Attention Deficit Hyperactivity Disorder are correlated with diminished dopamine activity. However, nucleic acid immunomodulators face challenges in biological systems due to poor cell permeability and rapid degradation by nucleases. Furthermore, nucleic acid immunoadjuvants may elicit innate immune system responses and potential off-target effects. Moreover, as standalone therapies, their efficacy as immunotherapeutic agents appears relatively modest.

Dopamine can be formulated into intravenous drugs, and although it may not penetrate the blood-brain barrier, its peripheral effects render it valuable in treating conditions like heart failure or shock, particularly in newborns. Moreover, studies have suggested that moderate dopamine levels can enhance the quality of sleep.

Glutamic acid (GA) is a naturally-occurring amino acid abundantly present in proteins. As it can be synthesized within the human body, it falls under the category of non-essential amino acids. This amino acid is widely distributed in both animal and plant organisms in diverse forms. Notably, it acts as the primary excitatory neurotransmitter in the nervous systems of vertebrates and serves as a precursor to gamma-aminobutyric acid (GABA).

Vitamin B6, also referred to as pyridoxine or the anti-dermatitis vitamin, belongs to the B-complex group of vitamins. Classified as an essential nutrient, it comprises six interconvertible vitamers closely associated with amino acid metabolism. Vitamin B6 functions as a coenzyme for enzymes participating in amino acid decarboxylation, transamination, and various metabolic processes. Pyridoxol represents a common chemical form of vitamin B6, while its biologically active form, pyridoxal phosphate, serves as a coenzyme in over 140 enzyme reactions crucial for amino acid, glucose, and lipid metabolism.

Plants have the capacity to synthesize pyridoxol as a defense mechanism against ultraviolet B radiation from sunlight and for the synthesis of chlorophyll. Conversely, animals lack the ability to synthesize vitamin B6 and must acquire it through dietary sources such as plants or other animals. While intestinal bacteria can produce a certain amount of vitamin B6, it is generally inadequate to fulfill the dietary requirements of animals. The recommended daily intake of vitamin B6 for adults ranges from 1.0 to 2.0 milligrams, with a safe upper limit falling between 25 to 100 milligrams per day.

Vitamin B6 deficiency is uncommon, with typical symptoms encompassing oral and ocular inflammation, drowsiness, and peripheral neuropathy, manifesting as altered sensation and impaired motor function in the extremities. Additional symptoms may comprise dermatitis, seizures, and anemia. Moreover, specific rare genetic disorders can precipitate infantile vitamin B6 deficiency and subsequent seizures.

Gastrodia elata, commonly referred to as Tianma, is a perennial saprophytic herb characterized by upright growth and rhizomatous tuberous roots. Renowned for its medicinal properties, Tianma holds a significant place in traditional Chinese medicine, notably documented in works such as the “Compendium of Materia Medica (Bencao Gangmu),” where it is lauded for its benefits to the liver meridian and qi circulation. The medicinal component of Tianma lies in its dried rhizome, which typically exhibits an elongated or cylindrical shape, often with wrinkles, and is distinguished by its distinct aroma, sweet taste, and subtle pungency. This herb is primarily found in regions such as Sichuan and Yunnan.

Black rice distinguishes itself by its deep purple or black hue. This rice variant possesses the ability to interbreed with other rice types without reproductive isolation, all falling under the broader category of rice. Historically, in ancient China, black rice earned the moniker “forbidden rice” as it was predominantly consumed by the privileged upper strata of society. Notably, the bran of black rice exhibits significantly higher activity of hydrophilic antioxidants compared to lipophilic ones. Anthocyanins and gamma-oryzanol, the primary constituents responsible for this antioxidant prowess, are predominantly concentrated in the inner layers of black rice bran. Extracts containing gamma-tocotrienol, a lipophilic antioxidant found alongside gamma-oryzanol, have displayed promising anti-inflammatory properties.

Wheat seedlings, commonly known as catgrass, denote the youthful shoots of plants within the Agropyron genus, notably Agropyron cristatum, a species akin to wheat. These tender leaves can be processed by juicing or dried and ground into powder. While the unprocessed plant contains a notable amount of indigestible cellulose, it also harbors beneficial constituents including chlorophyll, amino acids, vitamins, minerals, and enzymes. Within traditional Chinese medicine, Wheat seedlings is revered for its therapeutic potential. Notably, the “Compendium of Materia Medica” extols its virtues, citing its efficacy in treating various conditions: “Wheat sprouts possess a pungent, cold taste and are non-toxic. They are prescribed for ailments such as alcohol toxicity, sudden heat, alcohol-induced sores, and jaundice. . . . Extracting the juice and consuming it daily can alleviate thirst, mitigate chest heat, and promote regular bowel movements.” This underscores Wheat seedlings' anti-inflammatory and fever-reducing properties, its digestive support, and its role in intestinal regulation.

In the past, Tianma (Gastrodia elata) was predominantly utilized in extract form as an additive. Nevertheless, research has not substantiated the efficacy of isolated Tianma alone in effectively stimulating dopamine production or aiding in neural stability within the human brain. Consequently, supplementary components such as phosphatidylserine and vitamins are frequently incorporated to augment the stabilizing effects on brain function.

Given this context, there exists a pressing demand within the pertinent field to advance the development of Tianma fermentation complexes capable of authentically fostering dopamine production and facilitating neural stability in the brain. Experimental validation is imperative to ascertain their efficacy in retarding brain aging, safeguarding neurons, and promoting nervous system tranquility to enhance sleep quality.

### SUMMARY OF THE INVENTION

To address the issues mentioned above, this invention leverages natural ingredients blended in precise ratios and undergoes fermentation facilitated by probiotics. This innovative approach aims to elevate dopamine levels and augment the presence of antioxidant compounds such as SOD, GPx, and G6PD within brain tissues. Concurrently, it endeavors to diminish the levels of oxidative substances like 8-oxodG and MDA in brain tissues. The resultant formulation showcases promising potential in attenuating brain aging processes and providing robust protection for brain neuron.

In some embodiments, A method for preparing a fermentation complex with anti-aging and sleep-promoting effects through dopamine generation in the brain, comprising: a raw material crushing process, a negative pressure food processing process, an refinement Fermentation process, and a modification fermentation process.

In some embodiments, the raw material crushing process is obtaining crude extract by individually pressing raw material.

In some embodiments, raw material comprises Gastrodia elata fruiting body, Black rice, and Wheat seedlings.

In some embodiments, preferably, the crude extract comprises Gastrodia elata fruiting body extract, Gastrodia elata fruiting body residue, black rice extract, black rice residue, Wheat seedlings extract, and Wheat seedlings residue.

the negative pressure food processing process involves subjecting the crude extract to a pressure range of 20 cmHg to 60 cmHg for a duration spanning 5 to 14 days, consequently yielding a preliminary extract.

In some embodiments, the refinement fermentation process involves introducing 0.1% to 0.5% (w/w) of pectinase and inoculating 0.2% to 2% (w/w) of lactic acid bacteria into the initial extract. This refinement fermentation process maintains a fermentation temperature ranging from 22° C. to 28° C., allowing fermentation to proceed for a duration of 8 to 14 days, resulting in the production of a first fermentation liquid.

In some embodiments, the modification fermentation process involves introducing 0.2% to 2% (w/w) of yeast or acetic acid bacteria into the first fermentation liquid. This modification fermentation process maintains a fermentation temperature within the range of 22 to 28° C., continuing fermentation for 10 to 21 days to obtain a second fermentation liquid.

In some embodiments, the fermentation complex comprises the crude extract, the lactic acid bacteria, and the yeast or acetic acid bacteria.

In some embodiments, the crude extract includes Gastrodia elata fruiting body extract, Gastrodia elata fruiting body residue, black rice extract, black rice residue, Wheat seedlings extract, and Wheat seedlings residue.

In some embodiments, the fermentation complex enhances both the quantity and activity of lactic acid bacteria in the gastrointestinal tract of mammals, leading to the synthesis of gamma-aminobutyric acid (GABA).

In some embodiments, the fermentation complex improves individual sleep quality, maintaining the average duration of deep sleep within the normal range.

In some embodiments, the fermentation complex has the ability to maintain the percentage of rapid eye movement (REM) sleep in the overall sleep cycle within the range of 20% to 25%.

In some embodiments, the fermentation complex exhibits preventive and/or therapeutic effects against Parkinson's disease.

In some embodiments, the fermentation complex has the capability to enhance antioxidant effects in the brain.

In some embodiments, the fermentation complex possesses neuroprotective effects.

In some embodiments, the fermentation complex exhibits therapeutic effects on nerve injury.

In some embodiments, the fermentation complex has the efficacy to prevent a reduction in dopamine levels in the brain.

In some embodiments, the fermentation complex exhibits therapeutic effects on a reduction in dopamine levels in the brain.

### BRIEF DESCRIPTION OF THE DRAWINGS

FIG. 1 shows the sugar content variation among different strains during the refinement Fermentation process of the present invention.

FIG. 2 shows the pH variation among different strains during the refinement fermentation process of the present invention.

FIG. 3 shows the sugar content variation among different strains during the modification fermentation process of the present invention.

FIG. 4 shows the pH variation among different strains during the modification fermentation process of the present invention.

FIG. 5 shows the pH variation among different strains during the modification fermentation process of the present invention.

FIG. 6 shows the fermented complex, which promotes dopamine generation and has anti-aging and sleep-inducing effects, in delaying aging and promoting sleep through the generation of dopamine in the brain.

FIG. 7 shows the effect of the fermented complex, which promotes dopamine generation and has anti-aging and sleep-inducing effects, on delaying aging and promoting sleep through dopamine generation in the brain was evaluated on tyrosine hydroxylase brain tissue slices.

FIG. 8 shows the impact of the fermentation complex, which promotes dopamine generation and has anti-aging and sleep-inducing effects, on the activity of the antioxidant enzyme G6PD in brain tissue, contributing to the delay of aging and sleep-inducing effects.

FIG. 9 shows the effect of the fermentation complex, which promotes dopamine generation and has anti-aging and sleep-inducing effects, on the activity of the antioxidant enzyme GPx in brain tissue.

FIG. 10 shows the impact of the fermentation complex, which promotes dopamine generation and has anti-aging and sleep-inducing effects, on the activity of the antioxidant enzyme superoxide dismutase (SOD) in brain tissue.

FIG. 11 shows the effect of the fermentation complex, which promotes dopamine generation and has anti-aging and sleep-inducing effects, on the content of oxidative product 8-oxodG in brain tissue.

FIG. 12 shows the impact of the fermentation complex, which promotes dopamine generation and has anti-aging and sleep-inducing effects, on the content of lipid peroxidation product MDA in brain tissue.

### DETAILED DESCRIPTION OF THE INVENTION

### Definition

NC indicates Negative control; Ctrl indicates Control; L indicates Low dosage; M indicates recommended dosage; H indicates high dosage; F indicates female with high dosage.

In the picture * mark indicates a significant difference from the NC group; +mark indicates a significant difference from the Ctrl group; #mark #indicates a significant difference between the two groups.

### Example 1. The Preparation of the Fermentation Complex

The Fermentation complex is prepared through a staged fermentation process involving Gastrodia elata, black rice, and Wheat seedlings, combined with lactic acid bacteria, yeast, or acetic acid bacteria. The fermented liquid obtained after completion of fermentation is filtered and retained. The staged fermentation steps include:
(A) The raw material crushing process: Physically pressing Wheat seedlings into small fragments allows for the retention of juice, pulp, or leaf residues. Similarly, Gastrodia elata and black rice are softened by soaking in hot water at temperatures ranging from 70-100° C. for a duration of 30 minutes. Subsequently, they are pressed to form fragmented pieces, while ensuring the preservation of both the juice and residues.
(B) The negative pressure food processing process: Choose one or a combination of brown sugar (non-centrifugal Sugar), isomaltose, xylitol, granulated sugar, sugar, sucrose, thereof, add 0.2-1% (w/w) to augment the osmotic pressure of the extract. Concurrently, utilize a negative pressure extraction method within a vacuum environment ranging from 20 cmHg to 60 cmHg, sustaining the extraction process for a duration of 5-14 days. This method effectively ruptures the cell walls of vegetables and fruits, liberating intracellular nutrients and polysaccharides.
(C) The refinement fermentation process: Incorporate one or a combination of lactic acid bacteria, such as Lactobacillus plantarum, Lactobacillus delbrueckii, Lactococcus lactis, Lactobacillus acidophilus, or Bifidobacterium bifidum, into the extract at a ratio of 0.2-2% (w/w). Maintain the fermentation temperature within the range of 22-28° C. and continue fermentation for a period of 8-14 days. During this fermentation stage, exploit the inherent properties of lactic acid bacteria, including the production of various decomposing enzymes. These enzymes facilitate the breakdown of nutrients from fruits and vegetables into smaller molecules. Additionally, incorporate pectinase at 0.1-0.5% (w/w) to enhance the breakdown rate during fermentation.
(D) The modification fermentation process: Incorporate one or a combination of yeast or acetic acid bacteria, such as Saccharomycopsis fibuligera, Saccharomyces cerevisiae, Pichia fermentans, Schizosaccharomyces pombe, Candida hansenii, Acetobacter xylinum, or Acetobacter suboxydans, into the extract at a ratio of 0.2-2% (w/w). Maintain the fermentation temperature within the range of 22-28° C. and continue fermentation for a period of 10-21 days. During this fermentation stage, leverage the microbial decomposition properties to produce various decomposing enzymes, which break down polysaccharide components into smaller molecules. Consequently, the viscosity of the fermentation liquid decreases, and its fluidity increases. Upon completion of fermentation, the resulting product is a complex of fermented catgrass.

### Example 2. Comparison of the Phase Fermentation Test

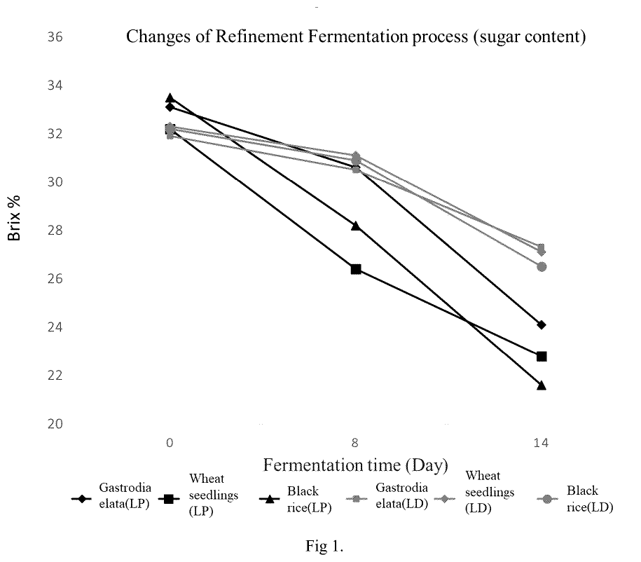

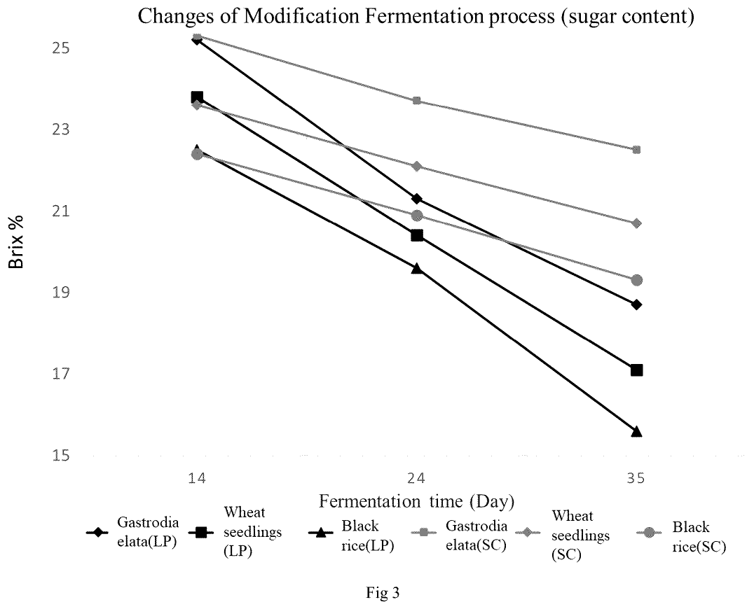

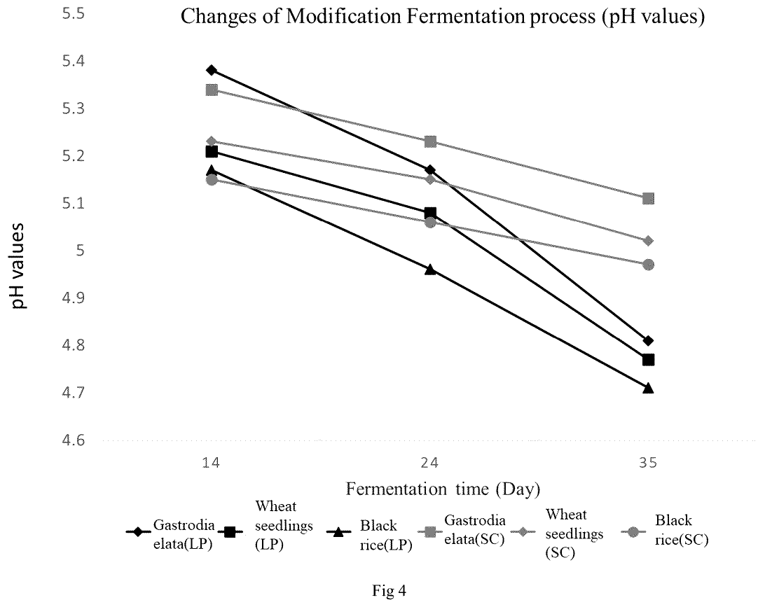

To explore the impact of various fermentation stages and strains on fermentation outcomes and achieve optimal fermentation results, different strains were employed for fermentation experiments across two stages. In this embodiment, two distinct strains were utilized in each stage: Lactic Acid Bacteria (L. plantarum, denoted as LP) and Bulgarian Lactic Acid Bacteria (L. delbrueckii, referred to as LD) in the Phase Fermentation stage; White Mould (S. fibuligera, designated as SF) and Brewing Yeast (S. cerevisiae, known as SC) in the modification fermentation process. This allowed for a comparative assessment to determine the most effective fermentation strains. Throughout the fermentation process, alterations in sugar content and pH value were found to be directly correlated with the fermentation activity of the strains. Notably, in short-term fermentation, the activity of the strains markedly hastened the completeness of the fermentation. The findings, as presented in Tables 1 and 2 and FIGS. 1 to 4, illustrate that different strains indeed exert a significant influence on fermentation outcomes. In the Purification Fermentation stage, Lactic Acid Bacteria (L. plantarum) demonstrated superior efficacy in enhancing the decomposition effect compared to Bulgarian Lactic Acid Bacteria (L. delbrueckii). Conversely, in the Modification fermentation process, White Mould (S. fibuligera) exhibited greater proficiency in reducing the viscosity of the fermentation liquid and enhancing its fluidity compared to Brewing Yeast (S. cerevisiae).

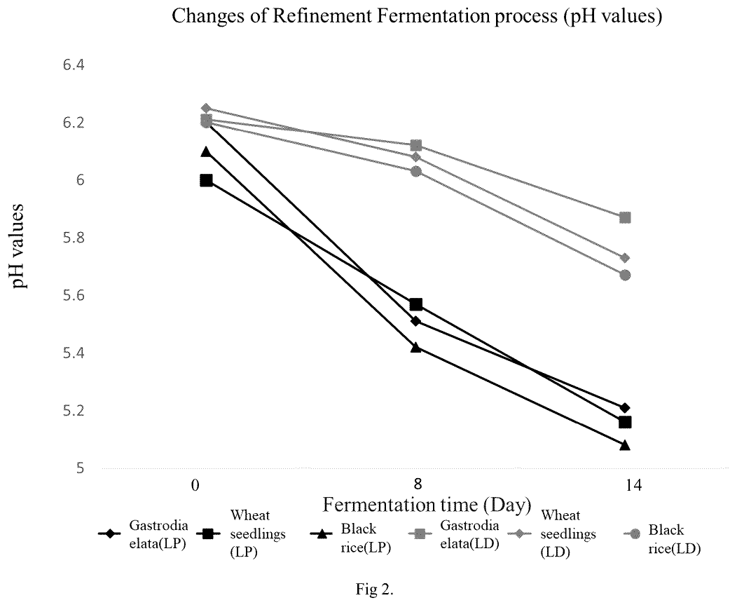

TABLE 1
Comparison of Differential Effects of Different Fermentation Strains in the refinement fermentation process

| Fermentation stage | | | | | | refinement fermentation process | | | | | | |
|---|---|---|---|---|---|---|---|---|---|---|---|---|
| Fermentation strain | | | | | | L. plantarum | | | | L. delbrueckii | | |
| | | | | | | Regulation of fermentation temperature to | | | | | | |
| | | | | | | enhance microbial proliferation activity without | | | | | | |
| | | | | | | adding carbon source, aiming to strengthen | | | | | | |
| | | | | | | component decomposition. pH decrease is | | | | | | |
| Fermentation effect | | | | | | significant during this stage | | | | | | |
| Fermentation time (day) | | | | | | 0 | 90 | 14 | | | 8 | 14 |
| Fermentation temperature (° C.) | | | | | | 22~28 | | | VS | 22~28 | | |
| Brix | Gastrodiaelata | Gastrodiaelata | Gastrodiaelata | Gastrodiaelata | Addition | 33.1 | 30.6 | 24.1 | | 31.9 | 30.5 | 27.3 |
| (FIG. 1.) | (LP) | (LD) | (SF) | (SC) | of | | | | | | | |
| | Wheat | Wheat | Wheat | Wheat | carbon | 32.2 | 26.4 | 22.8 | | 32.3 | 31.1 | 27.1 |
| | seedlings | seedlings | seedlings | seedlings | source | | | | | | | |
| | (LP) | (LD) | (SF) | (SC) | 0.2% | | | | | | | |
| | Black rice | Black rice | Black rice | Black rice | | 33.5 | 28.2 | 21.6 | | 32.2 | 30.9 | 26.5 |
| | (LP) | (LD) | (SF) | (SC) | | | | | | | | |
| pH | Gastrodiaelata | Gastrodiaelata | Gastrodiaelata | Gastrodiaelata | | 6.2 | 5.51 | 5.21 | | 6.21 | 6.12 | 5.87 |
| values | (LP) | (LD) | (SF) | (SC) | | | | | | | | |
| | Wheat | Wheat | Wheat | Wheat | | 6 | 5.57 | 5.16 | | 6.25 | 6.08 | 5.73 |
| | seedlings | seedlings | seedlings | seedlings | | | | | | | | |
| | (LP) | (LD) | (SF) | (SC) | | | | | | | | |
| (FIG. 2.) | Black rice | Black rice | Black rice | Black rice | | 6.1 | 5.42 | 5.08 | | 6.2 | 6.03 | 5.67 |
| | (LP) | (LD) | (SF) | (SC) | | | | | | | | |

TABLE 1
Comparison of Differential Effects of Different Fermentation Strains in the refinement fermentation process

| Fermentation stage | | | | | | refinement fermentation process | | | | | | |
|---|---|---|---|---|---|---|---|---|---|---|---|---|
| Fermentation strain | | | | | | S. fibuligera | | | | S. cerevisiae | | |
| | | | | | | Utilizing microbial decomposition characteristics | | | | | | |
| | | | | | | to produce various decomposition enzymes, breaking | | | | | | |
| | | | | | | down polysaccharide components into smaller molecules. | | | | | | |
| | | | | | | This results in a decrease in the viscosity and an | | | | | | |
| Fermentation effect | | | | | | increase in the fluidity of the fermentation broth | | | | | | |
| Fermentation time (day) | | | | | | 14 | 24 | 35 | | 14 | 24 | 35 |
| Fermentation temperature (° C.) | | | | | | 22~28 | | | VS | 22~28 | | |
| Brix | Gastrodiaelata | Gastrodiaelata | Gastrodiaelata | Gastrodiaelata | Addition | 25.2 | 21.3 | 18.7 | | 25.3 | 23.7 | 22.5 |
| (FIG. 1.) | (LP) | (LD) | (SF) | (SC) | of | | | | | | | |
| | Wheat | Wheat | Wheat | Wheat | carbon | 23.8 | 20.4 | 17.1 | | 23.6 | 22.1 | 20.7 |
| | seedlings | seedlings | seedlings | seedlings | source | | | | | | | |
| | (LP) | (LD) | (SF) | (SC) | 0.2% | | | | | | | |
| | Black rice | Black rice | Black rice | Black rice | | 22.5 | 19.6 | 15.6 | | 22.4 | 20.9 | 19.3 |
| | (LP) | (LD) | (SF) | (SC) | | | | | | | | |
| pH | Gastrodiaelata | Gastrodiaelata | Gastrodiaelata | Gastrodiaelata | | 5.38 | 5.17 | 4.81 | | 3.34 | 5.23 | 5.11 |
| values | (LP) | (LD) | (SF) | (SC) | | | | | | | | |
| (FIG. 2.) | Wheat | Wheat | Wheat | Wheat | | 5.21 | 5.08 | 4.77 | | 5.23 | 5.15 | 5.02 |
| | seedlings | seedlings | seedlings | seedlings | | | | | | | | |
| | (LP) | (LD) | (SF) | (SC) | | | | | | | | |
| | Black rice | Black rice | Black rice | Black rice | | 5.17 | 4.96 | 4.71 | | 5.15 | 5.06 | 4.97 |
| | (LP) | (LD) | (SF) | (SC) | | | | | | | | |

### Example 3. Establishment of Parkinson's Animal Model

Establishment of Parkinson's Model: Male mice were divided into four groups, including the MPTP-induced control group (Ctrl), MPTP-induced group with low-dose Ganoderma lucidum fermentation complex (L), MPTP-induced group with recommended dose Ganoderma lucidum fermentation complex (M), and MPTP-induced group with high-dose Ganoderma lucidum fermentation complex (H). Female mice comprised one group, which received MPTP induction and high-dose Ganoderma lucidum fermentation complex (F). Each group consisted of 12 mice. For groups receiving the Ganoderma lucidum fermentation complex, it was administered continuously via oral gavage for 28 days. The low-dose group received a dosage equivalent to 0.195 mg per gram of mouse body weight, corresponding to 250 mg/70 kg/day in humans. The recommended dose group received a dosage equivalent to 0.39 mg per gram of mouse body weight, corresponding to 500 mg/70 kg/day in humans. The high-dose group received a dosage equivalent to 1.17 mg per gram of mouse body weight, corresponding to 750 mg/70 kg/day in humans.

### Example 4. Intestinal Endogenous GABA Production Experiment

In a simulated intestinal environment with a pH of 8.3 using NaOH solution, probiotics were introduced to replicate intestinal bacterial and environmental conditions. Subsequently, 750 mg of the fermentation complex was added to a 50 ml NaOH aqueous solution. Samples were collected hourly to measure bacterial counts and GABA content. To compare the distinctions between the fermentation complex of the present invention and conventional Wheat seedlings extract, a parallel experiment was conducted by blending extracts of Wheat seedlings, black rice, and wheat seedlings in the same proportions as the fermentation complex. The outcomes are detailed in Table 3 and 4.

TABLE 3
The Results of the GABA Endogenous Production Experiment Simulating
the Intestinal Environment with the Fermentation complex

| | GABA contain | effect |
|---|---|---|
| Hour | mg/50 ml | — |
| 1 | 42.25 ± 1.1 | 750 mg of fermentation complex begins |
| | | enhancing the synthesis of GABA in the intestine |
| | | within the first hour |
| 2 | 79.35 ± 1.53 | — |
| 3 | 95.06 ± 5.36 | Promotes the increase in both the quantity and |
| | | activity of beneficial gut bacteria (such as |
| | | lactobacilli capable of synthesizing GABA) |
| 4 | 108.85 ± 5.73 | Synthesizing a large amount of GABA precursor, |
| | | containing various precursors with different |
| | | structures within the crystal |
| 5 | 120.44 ± 12.51 | — |
| 6 | 142.07 ± 8.93 | — |
| 7 | 195.56 ± 6.9 | — |
| 8 | 220.75 ± 11.23 | Continuously synthesizing for 8 hours, yielding |
| | | 200 mg of GABA within that time frame |

TABLE 4
Results of GABA Endogenous Production Experiment
Simulating the Intestinal Environment with Conventional
Fungal Rice Grass Extract Complex

| | GABA contain | effect |
|---|---|---|
| Hour | mg/50 ml | — |
| 0.5 | 78.57 ± 2.1 | The 750 mg of fermentation complex begins |
| | | entering the intestine and immediately starts |
| | | absorbing GABA within half an hour |
| 1 | 63.16 ± 1.32 | Once the intestine has absorbed all the GABA |
| | | and utilized it, the body's internal GABA |
| | | levels begin to decrease. |
| 1.5 | 50.21 ± 3.71 | — |
| 2 | 48.01 ± 3.13 | — |
| 2.5 | 36.19 ± 2.37 | — |
| 3 | 22.34 ± 2.03 | — |
| 3.5 | 10.11 ± 1.07 | — |
| 4 | 2.08 ± 0.32 | By the fourth hour, GABA is nearly depleted |
| | | within the body. |

Comparison of Table 3 and Table 4 reveals that the fermentation complex of the present invention, in a simulated intestinal environment, releases GABA precursors (fermentation type) that, through the action of intestinal probiotics, promote the endogenous production of GABA. When slowly released in the intestine for more than 3 hours, a substantial synthesis of GABA occurs in the fourth hour, representing the optimal time for the human body to enter deep sleep. This has a phased stress-relieving and sedative effect. GABA precursors slowly generate after crossing the blood-brain barrier, with a continuous production time of over 8 hours, ensuring that GABA is fully absorbed and utilized by the brain.

In contrast, conventional fermentation complex reaches their peak GABA content within 0.5 hours of ingestion due to absorption by the human body. However, with increasing time, the GABA content gradually decreases. This indicates that the conventional fungal rice grass extract complex cannot effectively promote the endogenous production of GABA in the human intestinal tract. Instead, due to the absorption process, the GABA content decreases over time until it is depleted. Therefore, the fermentation complex of the present invention and the non-fermented conventional fungal rice grass extract complex fundamentally differ in their nature.

### Example 5. Sleep Monitoring

Conducted on 18 individuals aged 20 to 60, encompassing both males and females, the study entailed the nightly administration of 750 mg of fermented compound of bacterial rice grass, administered 30 minutes prior to bedtime. Participants were outfitted with smart wearable devices to meticulously monitor their sleep patterns over a span of 5 days. This monitoring encompassed the tracking of sleep duration and the duration of the rapid eye movement (REM) phase. Research indicates that a 5% reduction in REM sleep is correlated with a 17% rise in mortality rates and an increased susceptibility to developing dementia.

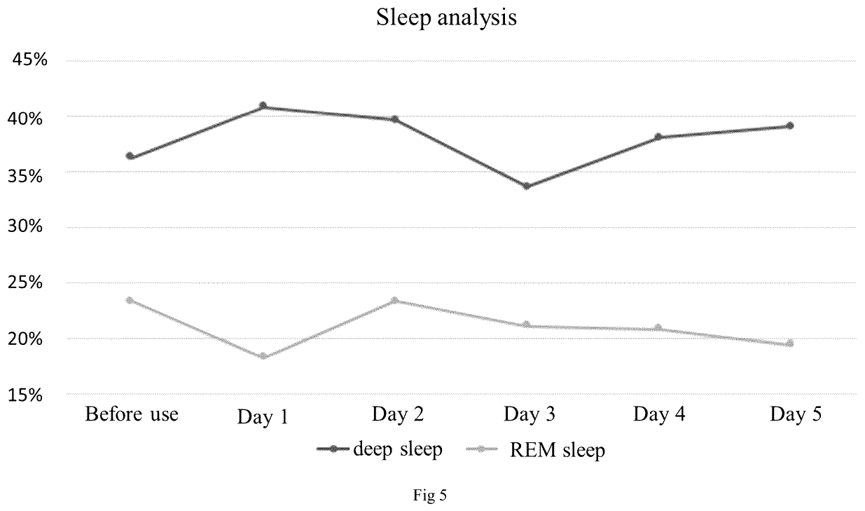

As depicted in FIG. 5, the results from the study indicate that on the first day of usage, there was an immediate increase in the deep sleep duration for all participants, with an average increment of 4.6%. Additionally, the rapid eye movement (REM) phase decreased by 5.09%. On the second day, there was a slight reduction in deep sleep duration, accompanied by a significant increase in REM time. This suggests that on the second day, bodily functions began to gradually recover, and there was an enhancement in brain activity, contributing to memory consolidation. From the third day onwards, the average deep sleep duration remained within the normal range, and the REM phase percentage stabilized between 20-25%, indicating a healthy sleep pattern.

### Example 6. Measurement of Dopamine Concentration in the Brain

Dopamine, primarily located in the brain regions of the human body and commonly referred to as the “happiness hormone,” plays a crucial role in regulating emotions and responding to stress. Moderate secretion of dopamine contributes to improved sleep.

The experiment induced a young Parkinson's disease mouse model using drugs, administering three different doses to male mice and the highest dose to female mice. After continuously providing the mice with the fermented complex of mushroom and rice grass for 28 days, the mice were sacrificed. Following the sacrifice, the striatum was extracted from the mouse brain to measure the dopamine concentration, analyzing the dopamine levels in the brain tissue.

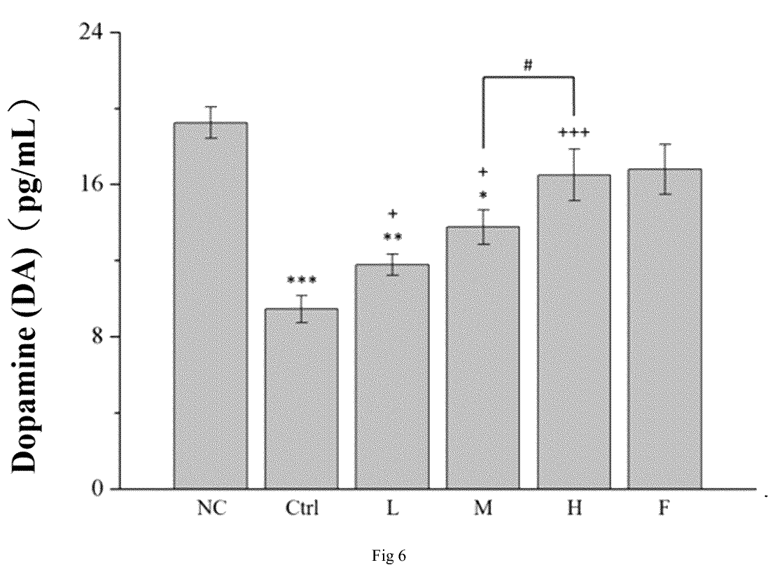

The results, as shown in FIG. 6, indicate a significant difference in mice with substantia nigra damage after low-dose and recommended-dose treatment, suggesting that both low and recommended doses have a significant therapeutic effect. However, there is still a notable difference compared to healthy mice, indicating that these doses can be evaluated for their preventive effects. In both male and female mice with substantia nigra damage, there was no significant difference from healthy mice after high-dose treatment, suggesting that the high dosage has an effective role in treating dopamine deficiency. In summary, the low and recommended doses demonstrate preventive effects against dopamine deficiency, while the high dosage can be considered an effective dosage for treatment.

### Example 7. Test of the Content of “Tyrosine Hydroxylase,” the Precursor of Dopamine in the Brain

Tyrosine hydroxylase (TH) serves as the foundation for dopamine secretion in brain tissues. A decline in TH concentration correlates with reduced dopamine production, potentially resulting in complications such as sleep disorders, mood swings, and difficulties in concentration.

The experiment induced a young Parkinson's disease mouse model using drugs, administering three different doses to male mice and the highest dose to female mice. Following continuous administration of the fermented mushroom grass complex for 28 days, the mice were euthanized. Brain tissues were collected, dehydrated, embedded, sliced, and stained. Immunostaining for Tyrosine hydroxylase (TH), an enzyme involved in dopamine synthesis, was conducted. Pathological alterations in TH expression were observed under an optical microscope. The abundance of TH served as a diagnostic and evaluative criterion

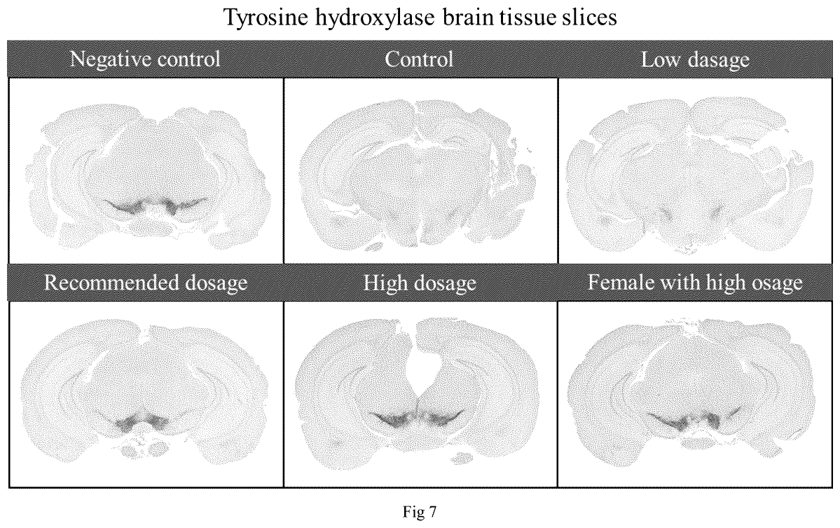

The results, as shown in FIG. 7, indicate that low-dose treatment manifests a discernible impact on mice with substantia nigra damage, indicating its efficacy in maintaining normal health and its potential for preventive use. In mice with substantia nigra damage treated with the recommended dose, a significant difference is observed, with a treatment effect notably superior to that of the low-dose group. However, compared to healthy mice, a significant difference persists. Conversely, in both male and female mice with substantia nigra damage, high-dose treatment exhibits no significant deviation compared to healthy mice, suggesting that the high dosage confers a preventive effect against dopamine deficiency. In summary, low-dose and recommended-dose treatments demonstrate preventive effects on substantia nigra damage, while a high dosage can be regarded as an effective treatment dose.

### Example 8. Antioxidant Indices in Blood

The aging of dopamine neurons is commonly considered to result from an excess of oxidative stress, leading to oxidative shrinkage of neurons. Therefore, the higher the antioxidant indices, the stronger the ability to protect neuronal cells. If the enzyme G6PD is present in brain tissue, it can generate antioxidants, resisting oxidative free radical damage. Studies have shown that patients with low G6PD enzyme activity lose their antioxidant capacity. SOD is a comprehensive free radical antioxidant enzyme, and the level of SOD activity can determine the strength of the antioxidant capacity in the organism. Hence, it is generally used as one of the indicators of antioxidant capacity.

The experiment induced aging in a Parkinson's disease mouse model through drug administration. Over a period of 12 weeks, the mice received three different doses, and blood samples were collected for the analysis of antioxidant indices, which encompassed the activities of SOD, G6PD, and GPx.

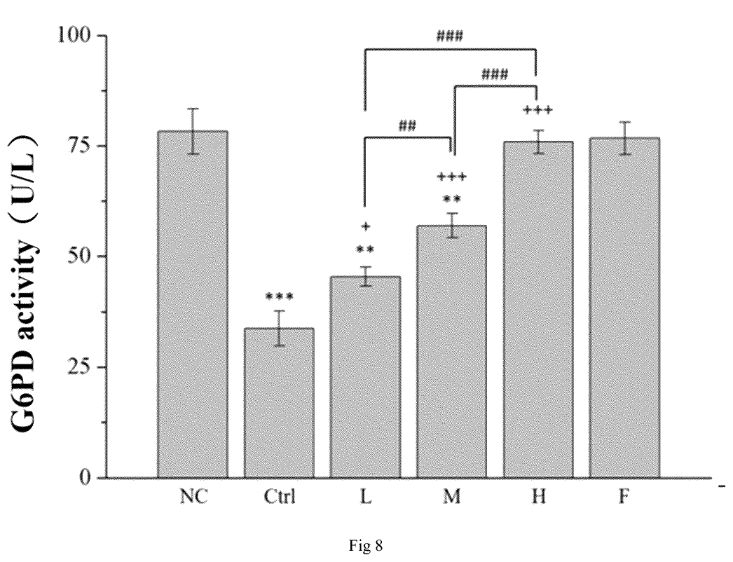

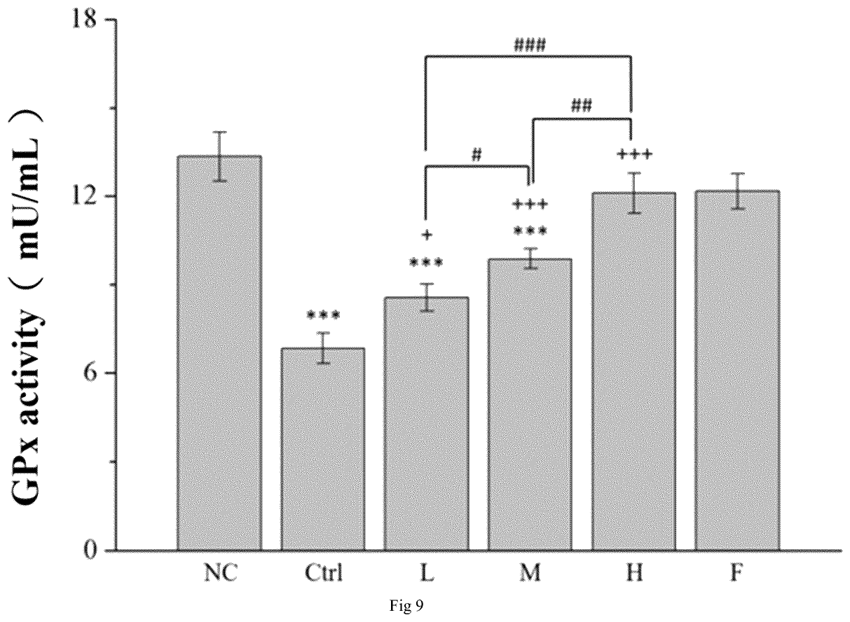

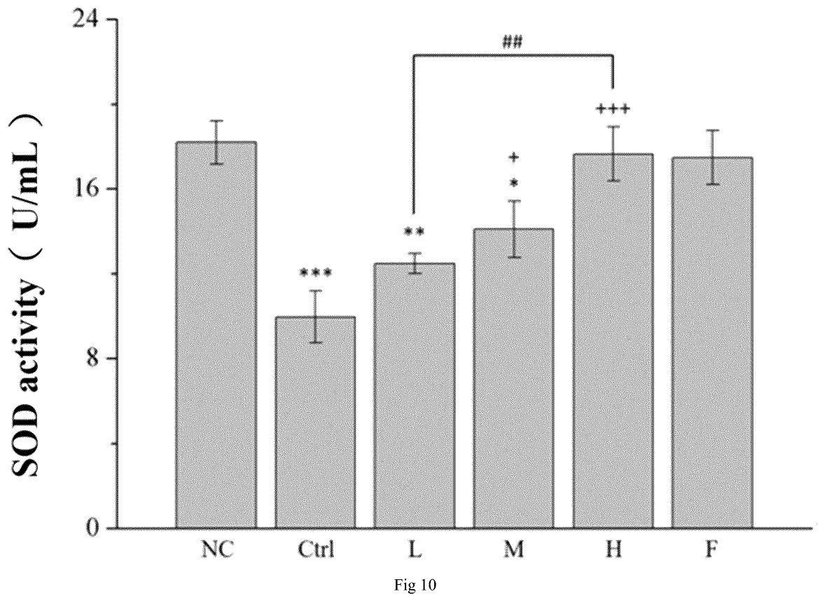

The results, as shown in FIGS. 8 to 10, reveal that regardless of the antioxidant indices, including G6PD, GPx, or SOD, the mice with substantia nigra damage exhibited significant differences after low-dose treatment. This indicates that low-dose treatment has a significant therapeutic effect, although it still differs significantly from the healthy mice. In mice with substantia nigra damage treated with the recommended dose, there is a significant difference, and the therapeutic effect is significantly higher than that of the low-dose group. However, there is still a significant difference compared to healthy mice. In both male and female mice with substantia nigra damage treated with a high dosage, there is no significant difference compared to healthy mice, indicating that a high dosage has a preventive effect on dopamine secretion deficiency. In summary, low and recommended doses have a preventive effect on dopamine deficiency and protect neurons. The high dosage can be considered an effective dose for treatment.

### Example 9. Measurement of Oxidative Substance Concentration in Brain Tissue

Research indicates that 8-oxodG, a potent free radical oxidative substance, can cause damage to dopaminergic neurons when present in the brain.

The experiment induced aging in a Parkinson's mouse model using drugs. Male mice received three different dosage levels, while female mice were administered the highest dosage. Following 28 days of continuous administration of fermented mushroom composite, the mice were euthanized. Brain tissues were collected, and mitochondria were isolated from these tissues. Subsequently, DNA was extracted from the mitochondria, and the content of 8-oxodG in brain mitochondrial DNA was analyzed to evaluate the extent of mitochondrial DNA damage.

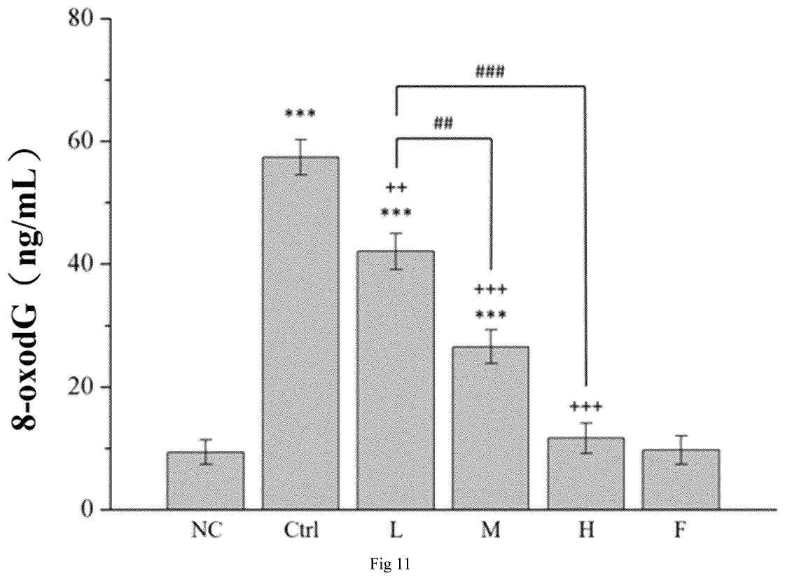

As shown in FIG. 11, the mice with substantia nigra damage treated with a low dosage exhibited significant differences, indicating that even a low dosage has a pronounced therapeutic effect. After treatment with the recommended dosage, there was a significant difference, and the therapeutic effect was significantly higher than that of the low-dosage group, but still exhibited significant differences compared to healthy mice. In male and female mice with substantia nigra damage treated with a high dosage, there was no significant difference compared to healthy mice, suggesting that a high dosage has a neuroprotective effect. In summary, low and recommended dosages have preventive effects against neuronal damage, while a high dosage can serve as an effective treatment for damaged neurons.

### Example 10. Measurement of Lipid Oxidation Concentration in Brain Tissue

Malondialdehyde (MDA) is a lipid peroxidation product, and its presence in the brain can lead to neuronal damage.

The experiment induced aging in a Parkinson's disease mouse model using drugs. Male mice received three different doses, while female mice were administered the highest dose. Following continuous administration of the fermented cordyceps complex for 28 days, the mice were euthanized, and brain tissue samples were collected. The analysis focused on measuring the levels of malondialdehyde (MDA), a product of lipid peroxidation. Reactive oxygen species (ROS) can induce the formation of MDA through lipid peroxidation, affecting cell membranes, lipoproteins, and other lipid-containing molecules, ultimately leading to oxidative shrinkage of neurons.

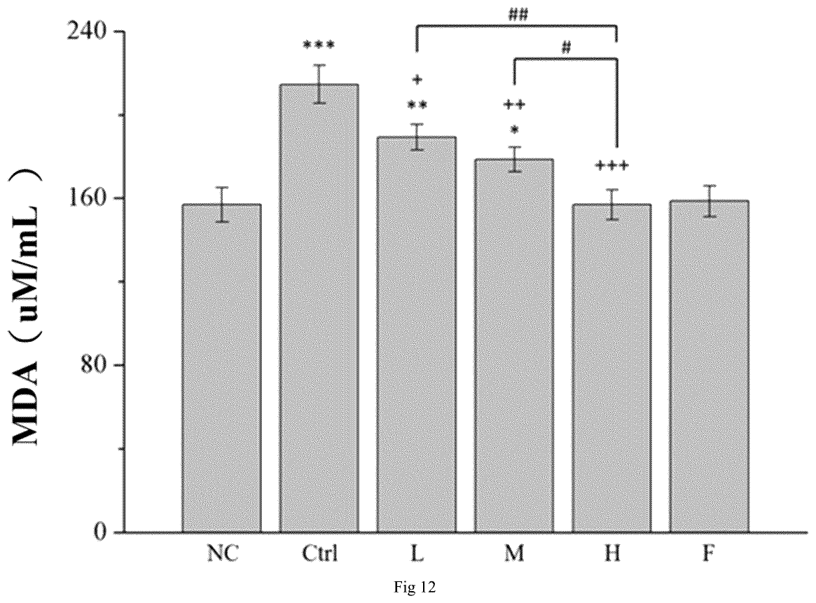

The results, as shown in FIG. 12, indicate that in the Parkinson's disease mouse model with damage to the substantia nigra, low-dose treatment exhibited significant differences, indicating a notable therapeutic effect. However, compared to healthy mice, there were still significant differences. After treatment with the recommended dose, there was a significant difference, and the therapeutic effect was significantly higher than that of the low-dose group, but there were still significant differences compared to healthy mice. In both male and female mice with substantia nigra damage, high-dose treatment showed no significant difference compared to healthy mice, suggesting a protective effect on neurons. In conclusion, low and recommended doses demonstrate a protective effect on neurons, while the high dosage can be considered an effective treatment dose for damaged neurons.

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims

1. A method for preparing a fermentation complex with anti-aging and sleep-promoting effects through dopamine generation in the brain, comprising:
a raw material crushing process: wherein the raw material is individually obtained by means of a pressing process to yield a crude extract, wherein the raw material is Gastrodia elata fruiting body, Black rice, and Wheat seedlings, wherein the crude extract includes Gastrodia elata fruiting body extract, Gastrodia elata fruiting body residue, black rice extract, black rice residue, Wheat seedlings extract, and Wheat seedlings residue;
a negative pressure food processing process: crude extract is subjected to a negative pressure food processing process at 20 cmHg to 60 cmHg for a duration of 5 to 14 days to obtain a first extract;
a refinement fermentation process: introduced a pectinase enzyme at 0.1% to 0.5% (w/w) and a lactic acid bacterium at 0.2% to 2% (w/w) into the first extract, in this refinement fermentation process, the fermentation temperature is maintained at 22 to 28° C., and fermentation continues for 8 to 14 days to obtain a first fermentation liquid; and
a modification fermentation process: introduced a yeast or acetic acid bacterium into the first fermentation liquid at 0.2% to 2% (w/w), and the fermentation temperature is maintained at 22 to 28° C., and fermentation continues for 10 to 21 days to obtain a second fermentation liquid.

2. The method of claim 1, wherein the raw material crushing process further comprises a Hot water treatment process, wherein the Gastrodia elata fruiting body, black rice, and Wheat seedlings are soaked in hot water at 70 to 100° C. for 30 minutes.

3. The method of claim 1, wherein Negative Pressure food processing process further comprises an increasing osmotic pressure process, wherein the increasing osmotic pressure process is involving 0.2 to 1% (w/w) of a sugar into the crude extract.

4. The method of claim 3, wherein the sugar comprises brown sugar (non-centrifugal Sugar), isomaltose, xylitol, granulated sugar, sugar, sucrose or a combination thereof.

5. The method of claim 1, wherein the lactic acid bacterium comprises Lactobacillus plantarum, Lactobacillus delbrueckii, Lactococcus lactis, Lactobacillus acidophilus, Bifidobacterium bifidum, or a combination thereof.

6. The method of claim 1, wherein the yeast or acetic acid bacterium comprises Saccharomyces fibuligera, Saccharomyces cerevisiae, Pichia pastoris, Schizosaccharomyces pombe, Candida utilis, Acetobacter xylinum, Acetobacter suboxydans, or a combination thereof.

7. The method of claim 1, wherein the method further comprises a granulation process, the second fermentation liquid is filtered and granulated by spray drying.

8. A fermentation complex with anti-aging and sleep-inducing effects, obtained by the method of claim 1, wherein the fermentation complex comprises the crude extract, the lactic acid bacteria, and the yeast or acetic acid bacteria, wherein the crude extract comprises Gastrodia elata fruiting body extract, Gastrodia elata fruiting body residue, black rice extract, black rice residue, Wheat seedlings extract, and Wheat seedlings residue.

9. A method for enhancing the quantity and activity of lactic acid bacteria capable of synthesizing gamma-aminobutyric acid (GABA) in the gastrointestinal tract of a mammal, wherein the method comprises administering to the subject the fermentation complex obtained by the method of claim 1.

10. The method of claim 9, wherein the method can enhance the synthesis of a precursor of gamma-aminobutyric acid (GABA) in the gastrointestinal tract of a mammal.

11. The method of claim 10, wherein the precursor of gamma-aminobutyric acid (GABA) maintains continuous production for at least 8 hours.

12. The method of claim 9, wherein the mammal selected from the group consisting of cat, dog, rabbit, cow, horse, sheep, goat, monkey, mice, rats, guinea pigs, hamsters, pigs, or humans.

13. A method for improving individual sleep quality, wherein the average duration of deep sleep remains within normal range, and the proportion of rapid eye movement (REM) sleep is restored to 20˜25%, the method comprises administering to the subject the fermentation complex obtained by the method of claim 1, wherein the effective dosage of the fermented complex is 250 mg/70 kg person/day to 750 mg/70 kg person/day.

14. A method for preventing Parkinson's disease, the method comprises administering to the subject the fermentation complex obtained by the method of claim 1, wherein the effective dosage of the fermented complex is 250 mg/70 kg person/day to 750 mg/70 kg person/day.

15. A method for treating Parkinson's disease, the method comprises administering to the subject the fermentation complex obtained by the method of claim 1, wherein the effective dosage of the fermented complex is 250 mg/70 kg person/day to 750 mg/70 kg person/day.

16. A method for enhancing brain antioxidant markers, the method comprises administering to the subject the fermentation complex obtained by the method of claim 1, wherein the effective dosage of the fermented complex is 250 mg/70 kg person/day to 750 mg/70 kg person/day.

17. A method for neuroprotection, the method comprises administering to the subject the fermentation complex obtained by the method of claim 1, wherein the effective dosage of the fermented complex is 250 mg/70 kg person/day to 750 mg/70 kg person/day.

18. A method for treating nerve injury, the method comprises administering to the subject the fermentation complex obtained by the method of claim 1, wherein the effective dosage of the fermented complex is 250 mg/70 kg person/day to 750 mg/70 kg person/day.

19. A method for preventing of decreased dopamine levels in the brain, the method comprises administering to the subject the fermentation complex obtained by the method of claim 1, wherein the effective dosage of the fermented complex is 250 mg/70 kg person/day to 750 mg/70 kg person/day.

20. A method for treating of decreased dopamine levels in the brain, the method comprises administering to the subject the fermentation complex obtained by the method of claim 1, wherein the effective dosage of the fermented complex is 250 mg/70 kg person/day to 750 mg/70 kg person/day.
